(19) United States
(12) Patent Application Publication
(10) Pub. No.: US 2024/0382869 A1
(43) Pub. Date: Nov. 21, 2024

(54) SPIN COLUMN

(71) Applicant: LABTURBO BIOTECH CORPORATION, TAIPEI CITY (TW)

(72) Inventors: Chi-Sheng TAI, TAIPEI CITY (TW); Shih Jeng DAI, TAIPEI CITY (TW); Shih Tsung HUANG, TAIPEI CITY (TW)

(21) Appl. No.: 18/661,828

(22) Filed: May 13, 2024

(30) Foreign Application Priority Data
May 17, 2023 (TW) 112118307

(51) Int. Cl. B01D 15/22

(52) U.S. Cl. CPC B01D 15/22

(57) ABSTRACT

The spin column of the present invention is used for filtering and extracting biological molecules, such as nucleic acids and proteins. When the spin column is used, it is placed inside a collection tube, which is then placed in a centrifuge slot and the centrifuge is activated. The internal volume of the spin column of the present invention is larger than that of a conventional spin column, allowing it to accommodate a larger amount of specimens. Additionally, the filter membrane positioned inside the spin column of the present invention is smaller than that of conventional spin columns. The amount of washing solution required to wash the biological molecules from the filter membrane is proportional to the size of the filter membrane. Therefore, less washing solution is required in the present invention. Since the spin column of the present invention can accommodate a larger amount of specimen, the number of biomolecules contained in the specimen is also larger. Since less washing solution is required to dissolve and recover the biomolecules from the filter membrane, the concentration of collected biological molecules in the washing solution is higher.

### FIELD OF THE INVENTION

The invention relates to a spin column, which is a device used for filtering and extracting biomolecules such as nucleic acids and proteins.

### BACKGROUND

To filter and extract biomolecules, such as nucleic acids and proteins, from a specimen, the specimen can be liquefied and then placed in a tube commonly known as a spin column. Filter membranes are inserted into the tube before injecting the specimen. When performing filtration extraction, the tube is placed into a collection tube, and then the collection tube is placed into the slot of a centrifuge, and then the centrifuge is started. Centrifugal force is thereby exerted on the specimen in the tube so that it passes through the filter membrane into the collection tube.

Due to the special material and pores of the filter membrane, when the sample passes through the filter membrane, the biomolecules in the sample to be extracted will be adsorbed on the filter membrane. The filter membrane is then cleaned with a cleaning solution to remove impurities in the membrane, and then the recovery solution is used to dissolve the biomolecules attached to the filter membrane and recover them to complete the extraction process.

The centrifuges mentioned above are small desktop centrifuges that have been used in laboratories for more than 30 years. They usually have 24 or 32 slots. Regardless of the brand, the slot specifications are roughly the same, with only a few millimeters of deviation. In the process of filtration and extraction, the centrifuge, spin columns, and collection tubes need to cooperate with each other. Since the specifications of the centrifuge slots are consistent among centrifuges, the specifications and shapes of the spin columns and collection tubes are also similar regardless of brand, differing by less than a few millimeters. The outer diameter of the spin column usually tapers from the opening to the bottom. Therefore, the size of the conventional spin column is roughly fixed, and its capacity to load the total volume of the specimen is also similar. The maximum suitable volume for operation is about 750 μl, and the elusion buffer is about 60 μl. However, in order to achieve more accurate test results, the higher the concentration of extracted biomolecules, the better. Therefore, it is necessary to put a larger amount of specimen into the spin column and reduce the volume of the elusion buffer to obtain higher concentration of biomolecules. However, as mentioned above, the sizes of conventional centrifuges, collection tubes and spin columns have been fixed, making it impossible to place a larger amount of specimens in the spin column. Therefore, it is necessary to develop new spin columns to obtain higher concentration of biomolecules that can also be used in conventional centrifuges.

### SUMMARY OF INVENTION

The volume of the spin column of the present invention can be greater than 1050 μl and the portion accommodating the filter membrane has a smaller diameter, so that the volume of the elution buffer can be reduced from 60 μl for the conventional spin column to 30 μl. Under the same conditions, the calculated concentration is 2.8 times that of a conventional spin column (=1050 μl/750 μl×60 μl/30 μl), which can effectively increase the concentration of biomolecules to meet the needs of subsequent applications such as biochemical assays.

The specific features of the spin column of the present invention are that it has three parts, namely upper, middle, and lower sections, the overall length is roughly the same as that of the conventional one, the outer diameter of the middle section is the same as that of the conventional one, the inner and outer diameters of the upper section are larger than the inner and outer diameters of the middle section, respectively, and the inner diameter of the lower section is smaller than that of the middle section. The lower section is provided with filter membranes of appropriate diameter. Furthermore, the length ratio of the three sections is also different from that of the conventional one, especially the length ratio between the upper section and the middle section, which is larger than that of the conventional one. Since the inner diameter of the upper section is larger than that of the middle section and the ratio of the length of the upper section to the length of the middle section is larger than that of the conventional one, the overall spin column has a larger volume than that of the conventional one.

The inner diameter of the lower section of the spin column of the present invention, where the filter membrane is placed, is smaller than that of the conventional spin column. Therefore, under the same filter membrane thickness, the volume of the filter membrane placed in the spin column of the present invention is smaller than that of the conventional spin column. Although the volume of the filter membrane is smaller, the number of adsorbed biomolecules does not change. Because the volume of the filter membrane is smaller, the amount of elution buffer required to dissolve and recover the biomolecules absorbed by the filter membrane is also less, which can effectively increase the concentration of recovered biomolecules, thereby improving the accuracy of subsequent applications such as biochemical assays.

### BRIEF DESCRIPTION OF THE DRAWINGS

FIG. 1 shows a conventional spin column.

FIG. 2 shows a conventional collection tube.

FIG. 3 shows the state in which the conventional spin column is inserted into the collection tube.

FIG. 4 shows a conventional centrifuge.

FIG. 5 shows the state of the collection tube with the spin column being inserted into the centrifuge.

FIG. 6 shows an embodiment of the spin column of the present invention.

FIG. 7 shows the state of one embodiment of the spin column of the present invention being inserted into a collection tube.

FIG. 8 shows another embodiment of the spin column of the present invention.

FIG. 9 shows the embodiment of the spin column of the present invention being inserted into a collection tube.

### DETAILED DESCRIPTION

The embodiments of the spin columns of the present invention are described hereinafter with reference to the drawings.

Since the spin columns of the present invention are improved to address the shortcomings of the conventional spin column, the characteristics of the spin column of the present invention can be best appreciated by first reviewing those of the conventional spin column 10.

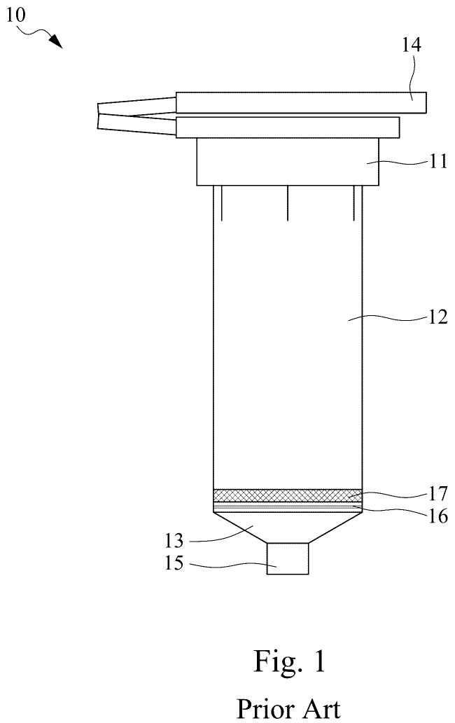

FIG. 1 shows a conventional spin column 10, which includes an upper section 11, a middle section 12, and a lower section 13, wherein an opening is formed on the top of the upper section 11 and may further include a cap 14 to close the opening tightly; the middle section 12 is a straight circular tube, which accommodates most of the specimen; the lower section 13 has an inverted cone shape, with an outlet 15 at the bottom, and the lower section 13 can accommodate the filter membrane 16 and a retaining ring 17. The retaining ring 17 secures the filter membrane 16 to the lower section 13.

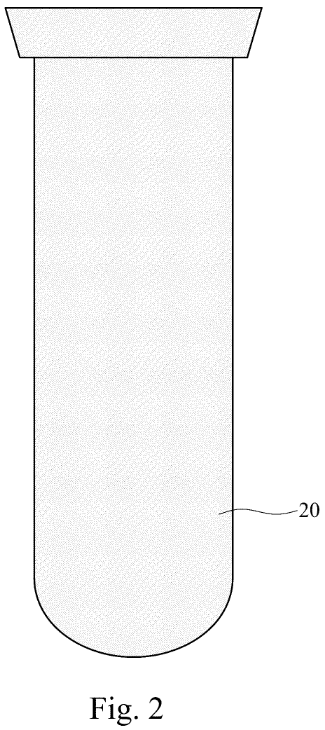

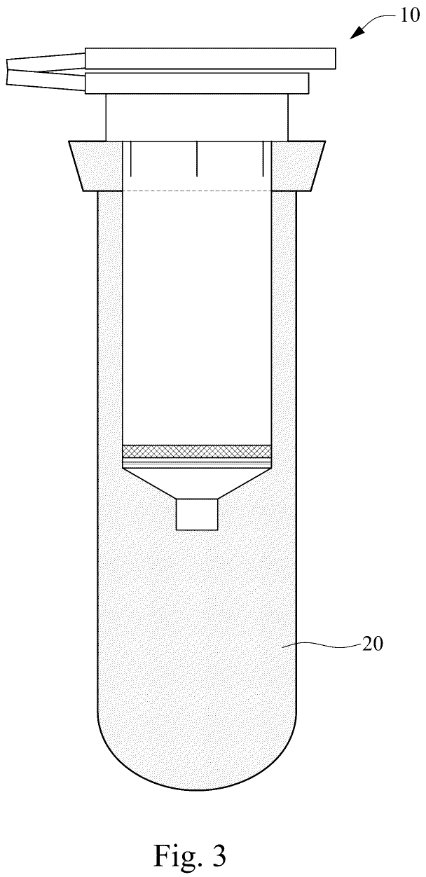

FIG. 2 shows a conventional collection tube 20, which has an open upper end and a closed lower end. FIG. 3 shows the state in which the conventional spin column 10 is inserted into the collecting tube 20, in which the upper section 11 of the spin column 10 is exposed above the collecting tube 20; the middle section 12 and the lower section 13 are accommodated in the collecting tube 20.

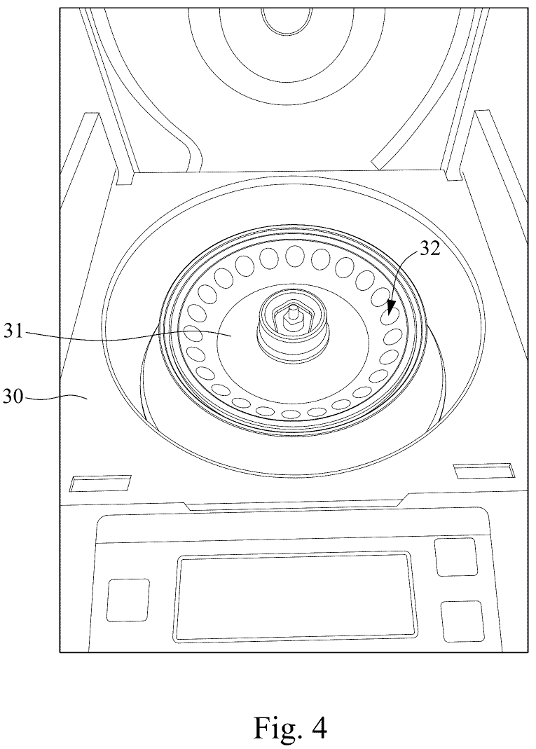

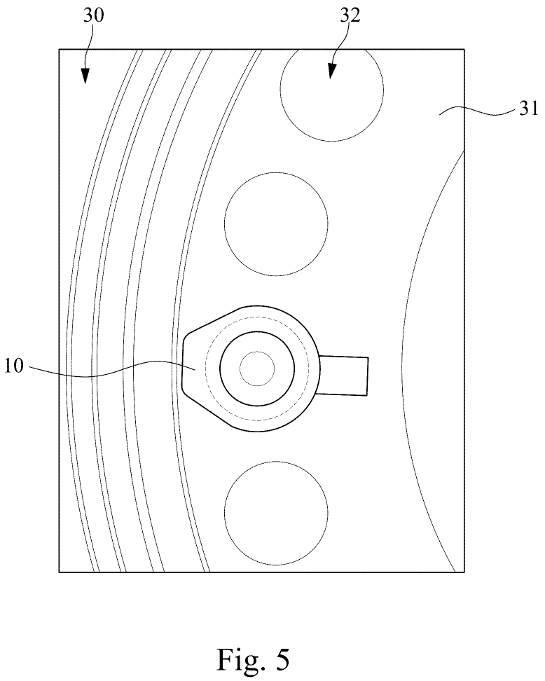

FIG. 4 shows a conventional centrifuge 30. The rotor 31 of the centrifuge 30 is evenly disposed with a plurality of slots 32 near the outer edge. FIG. 5 shows a state in which the collection tube 20 with the spin column 10 is inserted into the centrifuge 30. Only the upper section 11 of the spin column 10 is exposed outside the slot 32 of the centrifuge 30, while the other parts are hidden in the slot 32 of the centrifuge 30. After the centrifuge 30 is started, the specimen contained in the spin column 10 will pass through the filter membrane 16 and be received by the collection tube 20 due to the centrifugal force. The filter membrane 16 placed in the spin column 10 is made of a special material, and the biomolecules to be extracted from the specimen can be adsorbed by the filter membrane 16. After the elution process, the biomolecules on the filter membrane 16 can be dissolved out and received by the collection tube 20. When the concentration of biomolecules in the elution buffer is high, the accuracy of assay is also high. During the elution process, sufficient elution buffer needs to be added to dissolve and recover most of the biomolecules adsorbed by the filter membrane. Generally speaking, the amount of elution buffer added needs to be proportional to the size of the filter membrane.

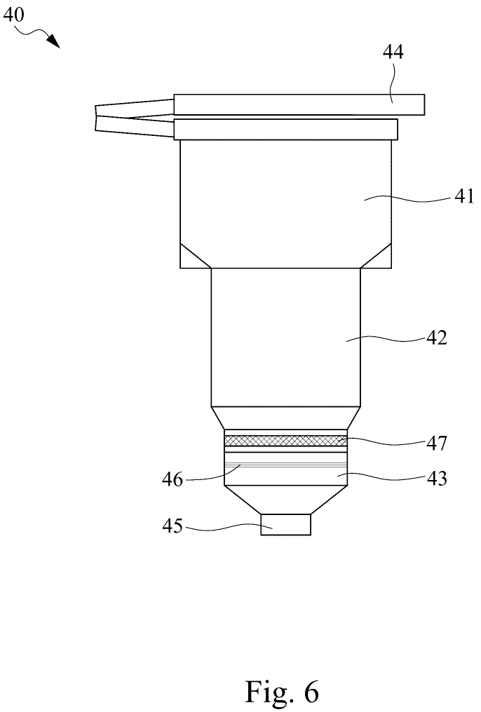

FIG. 6 shows an embodiment of the spin column 40 of the present invention. From comparison of the conventional spin column 10 in FIG. 1 with the spin column 40 of the present invention shown in FIG. 6, it can be understood that the spin column 40 can also be divided into three sections, i.e., upper, middle, and lower sections 41, 42, and 43. Among them, the outer diameter and inner diameter of the middle section 42 are respectively equivalent to the outer diameter and inner diameter of the middle section 12 of the conventional spin column 10; the outer diameter and inner diameter of the upper section 41 are respectively larger than the outer diameter and inner diameter of the middle section 42, and an opening is formed at the top, and can further include a cap 44 tightly covering the opening; the outer diameter and inner diameter of the lower section 43 are smaller than the outer diameter and inner diameter of the middle section 42, respectively. There is an outlet 45 at the bottom of the lower section 43, and the lower section 43 can accommodate a filter membrane 46 and a retaining ring 47. The retaining ring 47 secures the filter membrane 46 to the lower section 43.

The sum of the lengths of the upper, middle, and lower sections 41, 42, and 43 of the spin column 40 is approximately the same as the length of the upper, middle, and lower sections of the conventional spin column 10, between 28.0 mm and 30.0 mm; the length of the upper section of the spin column 40 is greater than the length of the upper section of the conventional spin column 10, between 9.5 mm and 10.5 mm; the length of the middle section of the spin column 40 is less than the length of the middle section of the conventional spin column 10, between 11.0 mm and 12.0 mm; the length of the lower section of the rotating tube 40 is similar to the length of the lower section of the conventional spin column 10, between 7.5 mm and 8.5 mm. Since the length of the upper section of the spin column 40 is greater than the length of the upper section of the conventional spin column 10, the volume of the spin column 40 is larger than that of the conventional spin column 10. Taking the spin column shown in FIG. 6 as an example, the volume of the spin column can be increased from the conventional 750 μl to 1050 μl, which is 1.4 times the conventional volume.

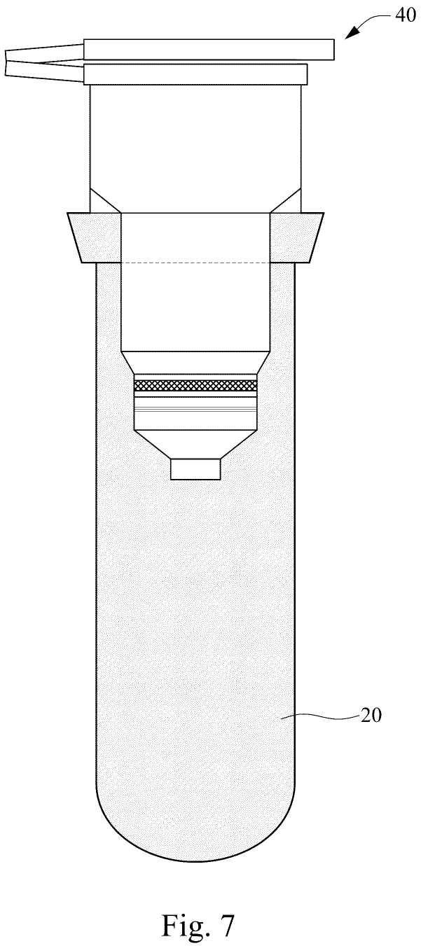

FIG. 7 shows an embodiment of the spin column 40 of the present invention being inserted into the collecting tube 20, in which the upper section 41 is exposed above the collecting tube 20; the middle section 42 and the lower section 43 are contained in the collection tube 20.

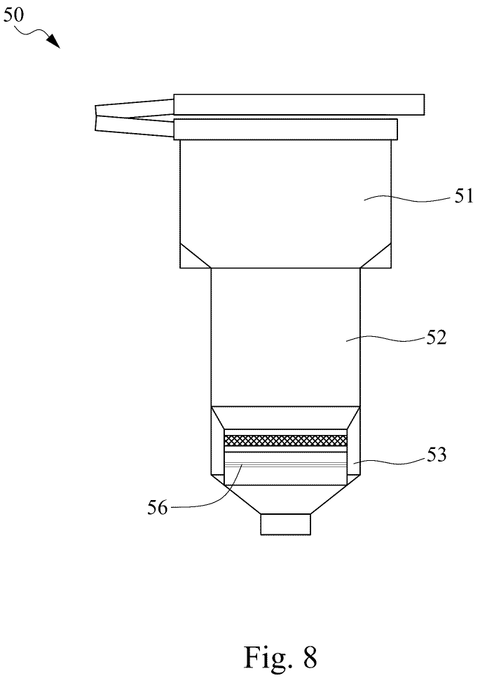

FIG. 8 shows another embodiment of the spin column 50 of the present invention. The difference from the embodiment of the spin column 40 of the present invention shown in FIG. 6 lies in the outer diameter of the lower section. The outer diameter of the middle section 52 may be the same as or slightly smaller than that of the lower section 53, and be larger than the outer diameter of the lower section 43 of the spin column 40. The inner diameter of the lower section 53 of the spin column 50 is still the same as that of the lower section 43 of the spin column 40.

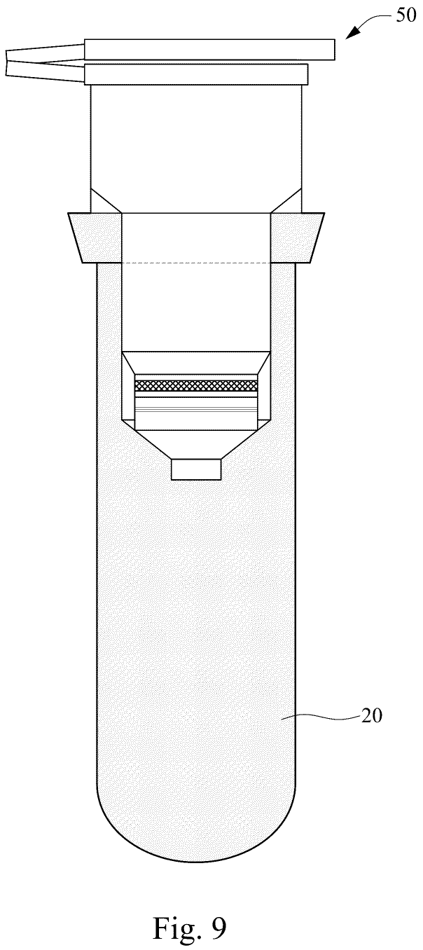

FIG. 9 shows the state in which the spin column 50 of the present invention is inserted into the collecting tube 20, in which the upper section 51 of the rotating tube 50 is exposed above the collecting tube 20; the middle section 52 and the lower section 53 are accommodated in the collecting tube 20.

The inner diameter of the lower section 43, 53 of the spin column 40, 50 of the present invention is smaller than that of the lower section 13 of the conventional spin column 10. Therefore, the diameter of the filter membrane 46, 56 placed in the lower section 43, 53 is also smaller than that of the filter membrane 16 placed in the lower section 13 of the conventional spin column 10. The inner diameter of the lower section 13 of the conventional spin column 10 is about 8.2 mm; the inner diameter of the lower sections 43, 53 of the spin column 40, 50 of the present invention can be between 6.5 mm and 5.5 mm. Under the same filter membrane thickness, the volume of the filter membrane 46, 56 is also smaller than that of the filter membrane 16. The appropriate volume of recovery solution for the spin column can be reduced from the conventional 60 μl to 30 μl.

When biological sample analysis is performed, correct analysis results often cannot be obtained because the obtained biological sample does not reach a sufficient concentration. Using the spin column of the present invention can obtain higher concentration of biomolecules, which is of great help in obtaining correct analysis results.

The content above describes only certain possible ways to implement the present invention. Modifications, replacements, and combinations of the above-mentioned embodiments can be easily completed by those skilled in the art in the field of the present invention and are within the scope of the inventive concept.

What is claimed is:

1. A spin column including:
an upper section, the top of which forms an opening, wherein the upper section has an outer diameter and an inner diameter;
a middle section, continuing from the upper section, having an outer diameter and an inner diameter, the outer diameter of the middle section being smaller than the outer diameter of the upper section and the inner diameter of the middle section being smaller than the inner diameter of the upper section; and
a lower section, continuing from the middle section and having an outer diameter and an inner diameter, the inner diameter of the lower section being smaller than the inner diameter of the middle section, and the bottom end of the lower section having an outlet.

2. The spin column of claim 1, wherein a cap is provided on the top of the upper section to tightly cover the opening.

3. The spin column of claim 2, wherein the outer diameter of the lower section is the same as the outer diameter of the middle section.

4. The spin column of claim 2, wherein the inner diameter of the lower section is between 6.5 mm and 5.5 mm.
